(19) United States
(12) Patent Application Publication
(10) Pub. No.: US 2004/0067967 A1
(43) Pub. Date: Apr. 8, 2004

(54) Treatment of urinary dysfunction

(76) Inventors: Julian Alexander Barden, New South Wales (AU); Angus Gidley-Baird, New South Wales (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/363,513

(22) PCT Filed: Aug. 28, 2001
(86) PCT No.: PCT/AU01/01079

(30) Foreign Application Priority Data
Aug. 28, 2000 (AU) PQ 9687

(51) Int. Cl. A61K 31/52

(52) U.S. Cl. 514/263.4; 514/263.24

(57) ABSTRACT

The invention provides a pharmaceutical composition for treatment or prevention of urinary dysfunction in a mammal. The composition includes a pharmaceutically effective amount of one or more substances capable of enabling the bladder of the mammal to mimic conditions found in advanced pregnancy. Alternately, the pharmaceutical composition has a pharmaceutically effective amount of one or more substances adapted to regulate the expression of one or more ATPases that control the supply of ATP to P2X receptors in the bladder of the mammal. In one aspect, the pharmaceutical composition may downregulate expression of subtype receptors P2X₁, P2X₂, P2X₃ and P2X₅, possibly upregulating expression of subtype receptors P2X₄ and P2X₆.

### TECHNICAL FIELD

This invention relates to treatment and/or prevention of continence problems, including urinary incontinence attributable to benign prostate hyperplasia in males and detrusor instability or sensory urgency in females and males. In particular, the invention can be useful for treatment of refractory cases; however the invention is not necessarily limited to this application. The invention is particularly concerned with incontinence in humans but, once again, is not necessarily limited thereto.

### BACKGROUND ART

Urinary incontinence is recognised as a problem having social and economic effects for both men and women. Either sex can suffer from instability of the detrusor muscle of the bladder or from sensory urgency. In men, benign prostate hyperplasia can lead to urinary incontinence.

Attempts have been made to treat or prevent urinary incontinence, especially in women. There have been several studies of the effects of estrogen therapy on postmenopausal women and most studies indicate that estrogen therapy in the form of hormone replacement therapy can alleviate some symptoms in some subjects.

However, estrogen treatment as disclosed in the prior art does not provide sufficient improvement in many subjects, especially in those cases considered refractory. The search has continued for a solution to the problem of urinary incontinence, as illustrated, for example, by U.S. Pat. No. 5,789,442 (K. Chwalisz & R. E. Garfield, assigned to Schering A G).

### BACKGROUND OF THE INVENTION

Part of the basis for the invention is found in research into purinergic receptor subtypes (P2X) in the bladder. It is known that P2X binding sites are present in the human bladder and it has been possible to detect the distribution of the P2X receptors in tissue. Subtypes P2X₁ to P2X₇ have been identified.

Further studies have been carried out in respect to patients suffering either from instability of the detrusor muscle of the bladder (“detrusor instability” or “DI”) or from sensory urgency (“SU”). The patients of particular interest were considered refractory, in that these patients had been tested urodynamically for confirmation of their condition; the patients were placed on at least two different anti-muscarinic drugs for at least one year, and at the same time underwent bladder training, without effect. The studies showed that, in the case of detrusor instability, there was clear evidence of a down-regulation of receptor subtypes P2X₃ and P2X₅, with the minor subtypes P2X₄, P2X₆ and P2X₇ exhibiting increased subsynaptic distribution. It may be a combination of the downregulation of the P2X₃ and P2X₅ subtypes with a small increase in overall distribution of the P2X₄, P2X₆ and P2X₇ subtypes that leads to an overall prolongation of purinergic response seen in the idiopathic detrusor instability detrusor. In the case of patients with sensory urgency, the studies showed clear evidence of a general down-regulation of all subtypes beneath the parasympathetic varicosities, except for P2X₇, which remained at low levels.

In these studies, a patient with sensory urgency typically had a first desire to void at less than 150 mL and maximum functional cystometric capacity at 150-300 mL and often even much lower. Infection as a cause of bladder incontinence in these patients was excluded as all showed negative mid-stream urine microscopy and culture. Unstable detrusor contractions were absent. Patients with idiopathic detrusor instability typically had a first desire to void at 150-200 mL and possessed a maximum cystometric capacity of 350-400 mL. Diagnosis of DI was made when detrusor contractions were observed on urodynamic testing and there was no outflow obstruction or neurological disease.

During pregnancy, there is usually increased pressure on the bladder. This is particularly the case during late pregnancy. Investigations have been made to determine the expression of P2X receptor subtypes during pregnancy.

It has been found that, in the pregnant rat bladder, some P2X receptor subtypes are down-regulated while others are up-regulated during pregnancy. In particular, it was found that the fast ionotropic subtypes P2X₁, P2X₂, P2X₃ and P2X₅ are progressively down-regulated from beneath the varicosities, whereas the slower subtypes, P2X₄ and P2X₆, are dramatically up-regulated by day 17 of the rat pregnancy. It is postulated that in humans these changes occur under the influence of pregnancy hormones, such as progesterone. Perhaps these hormones induce their biological effects by binding to cytoplasmic receptor proteins that transport hormone to the nucleus; subsequent interaction with DNA in the nucleus may modulate the gene expression for such proteins as the P2X receptors. It is also possible that, alternately or additionally, the hormones may affect the supply of ATP acting on the P2X receptors by reducing the expression of ATPases that control the supply of ATP to the receptors, with the fast types P2X₁, P2X₂, P2X₃ and P2X₅ being down-regulated, perhaps through mechanisms including receptor internalisation, leaving the slow types P2X₄ and P2X₆ up-regulated in contrast.

It is believed that the fast subtype P2X₇ may also be upregulated, along with P2X₄ and P2X₆ but to a lesser extent.

Accordingly, the approach of the present invention is found in the modulation of expression of the P2X receptors by mimicking conditions of advanced pregnancy, in which the pregnancy hormones are able to modify the expression of the P2X receptors in a pattern which acts to reduce the micturition initiation response, while ensuring enhanced emptying of the bladder. It is believed that the urination initiation signal is somehow desensitised in advanced pregnancy, while the capacity to properly empty the bladder is maintained, possibly through increasing the non-densensitising receptors, mainly P2X₄ and P2X₆. It is further believed that the invention relates in particular to treatment of those conditions which are refractory cases involving disruption to the purinergic receptor subtypes referred to above.

A factor in male incontinence may be partial occlusion of the prostatic urethra caused by hyperplasia. The approach of the invention involving manipulation of the P2X receptor subtype expression in the bladder to control the effects of incontinence has now been applied to the prostate to control hyperplasia. It has been found that the application of hormones, especially phytoestrogens and/or isoflavones of various combinations, in amounts of approximately 40 mg/day of active ingredient, can reduce prostatic bulk in humans associated with benign prostatic hyperplasia, thereby improving urinary function.

Phytoestrogen and/or isoflavone supplementation can also alleviate the symptoms of incontinence in patients generally, primarily women, but not confined to them.

While indications are that urinary incontinence is most prevalent in post-menopausal human females, it is to be understood that the invention is not limited to this but may also be applicable to other human females, to human males, and to other mammals.

Because current studies and investigations may not fully explain the working of the invention, it is necessary to define the invention in a number of aspects, as set out below. It is possible and likely that there will be overlap of at least some of those aspects.

### DISCLOSURE OF THE INVENTION

Accordingly, in a first aspect, the invention provides a pharmaceutical composition for treatment or prevention of urinary dysfunction in a mammal having lowered purinergic activation, causing partial loss of inhibition of muscaranic receptors, the composition including a pharmaceutically effective amount of one or more substances capable of enabling the bladder of the mammal to mimic conditions found in advanced pregnancy.

In a second aspect, the invention provides a pharmaceutical composition for treatment or prevention of urinary dysfunction in a mammal having lowered purinergic activation, causing partial loss of inhibition of muscaranic receptors, the composition including a pharmaceutically effective amount of one or more substances adapted to regulate the expression of one or more ATPases that control the supply of ATP to P2X receptors in the bladder of the mammal. Preferably, the ATPases control the local supply of ATP to the P9X receptor so as to down-regulate expression of receptor subtypes P2X₁, P2X₂, P2X₃ and P2X₅ in the bladder of the mammal at parasympathetic nerve neurotransmitter release sites. Preferably or alternately, expression of subtype receptors P2X₄ and PX2₆ is upregulated.

In a third aspect, the invention provides a pharmaceutical composition for treatment or prevention of urinary dysfunction in a mammal having lowered purinergic activation, causing partial loss of inhibition of muscaranic receptors, the composition including a pharmaceutically effective amount of one or more substances adapted to down-regulate expression of subtype receptors P2X₁, P2X₂, P2X₃ and P2X₅ in the bladder of the mammal at parasympathetic nerve neurotransmitter release sites. Preferably or alternately, expression of subtype receptors P2X₂ and P2X₄ is upregulated.

In a fourth aspect, the invention provides a pharmaceutical composition for treatment or prevention of urinary dysfunction in a mammal having lowered purinergic activation, causing partial loss of inhibition of muscaranic receptors, the composition including a pharmaceutically effective amount of one or more substances adapted to down-regulate expression of subtype receptors P2X₁, P2X₂, P2X₃ and P2X₅ while up-regulating expression of subtype receptors P2X₄ and P2X₆ in the bladder of the mammal at parasympathetic nerve neurotransmitter release sites.

In the various aspects of the invention above, the substances may include one or more pregnancy hormones. Preferably, the pregnancy hormones are chosen from the group consisting of progestins and estrogens. Progesterone may be mentioned as one preferred substance.

The substance (or substances, if more than one) may include one or more phytoestrogens and/or isoflavones. Phytoestrogens and/or isoflavones may be used in various combinations, as indicated above.

The quantity of the substance or substances, if more than one, depends on the mammal and the result to be achieved, preferably while limiting side effects. For example, in the case of humans, when enabling the bladder to mimic conditions found in advanced pregnancy, it is desirable to provide the substance or substances in sufficient amounts to increase the level of plasma progesterone tom 25 to 125 mg/mL of plasma. This can be achieved, for example, via oral administration or via implant with doses of up to 250 mg/day should exceed 5 mg/day.

The same doses may alter the receptor expression in DI and SU patients by down-regulating expression of receptor subtypes P2X₁, P2X₂, P2X₃ and P2X₅ in the bladder of the patient at parasympathetic nerve neurotransmitter release sites. Where expression of these receptor subtypes is already low as fund in SU patients, further down-regulating may in fact be minimal. These doses may also up-regulate expression of subtype receptors P2X₄ and P2X₆, as well as P2X₇ but to a lesser extent. Indeed, in both DI and SU patients this is likely to be the preferred outcome.

Phytoestrogen is conveniently provided by, for example, the commercially available product Promensil, manufactured by Novogen. A suitable amount may be in the range of 40-160 mg/day of Promensil. The range of 40-160 mg/day of Promensil can alleviate the symptoms of incontinence in female patients. When treating male patients, the dose of Promensil is preferably around 40 mg/day.

The substances are not limited to progestins, estrogens and phytoestrogens and/or isoflavones. While progesterone and phytoestrogens and/or isoflavones are preferred, this invention covers other substances or combination of substances which may be suitable. Especially in the case of human patients, appropriate combinations should be tried on a case-by-case basis, to optimise the desired effects while limiting any side effects in patients susceptible to side effects for reasons of sensitivity, for example.

There may-be a synergistic effect between progesterone and suitable corticosteroids such as desoxycorticosterone. In particular, the ratio of pregnancy hormones estrogen and progesterone and minerelocorticoids may be particularly important. Further, the pattern of use of one or more of the above pharmaceutically effective agents may need to be altered for optimum effect.

The invention also provides a method of treating or preventing urinary dysfunction in a mammal, including administering to the mammal a pharmaceutical composition as defined in any of the aspects above.

The invention also provides the use of a pharmaceutical composition defined in any of the aspects above, in the treatment or prevention of urinary dysfunction in a mammal.

Preferably, the mammal is a human.

### EXAMPLES OF THE INVENTION

The invention will now be illustrated by certain non-limiting examples thereof as follows:

### Example 1

Eighteeen female human patients with DI, aged from 30 to 81 years, were tested urodynamically. These tests revealed the first desire to void occurred at an average 173 mL (range 50-350 mL). The average maximum bladder capacity was 340 mL (range 150-570 mL). The average maximum detrusor pressure was 48 cm H₂O (range 18-100 cm H₂O).

Microscopic observation failed to reveal any SV2-labelled neurotransmitter release sites at parasympathetic nerve varicosities that were colocalized with either of the subtype receptors P2X₃ or P2X₅. The expression or synthesis of these two subtypes appeared markedly reduced in the detrusor from DI patients, compared with 22 adult control bladders.

In the DI patients, in the unstable muscle, P2X₄ and P2X₆ subtypes were more commonly associated with SV2-staining varicosities than in control bladders (36% and 33% versus 16% and 18%, respectively), but like the control bladders, image analysis showed that the intensity of the Cy2 fluorescence with the subtypes was low compared with P2X₁ and P2X₂ (<10%). The majority of SV2-labelled varicosities from DI patients were immunolocalized with trace amounts of P2X₇ compared with the lower levels found in control bladders. The levels observed were typically much lower than the levels observed in varicosities colocalized with P2X₁ and P2X₂.

Treatment: to a non-pregnant female human suffering from DI, administer progesterone in the amount of 50-250 mg/day to alleviate symptoms.

### Example 2

A study was conducted using rats fed either a phytoestrogen free diet for six months or an identical diet but supplemented with red clover (5%) as an example of a typical phytoestrogen enriched diet. The bladders of the rats fed the phytoestrogen free diet were indistinguishable from normal bladders in terms of P2X receptor expression. The rats fed the red clover supplement showed very clear signs of reduction in P2X₁, P2X₂, P2X₃ and P2X₅ expression with concomitant increase in P2X₄, and P2X₆ at subsynaptic loci.

Treatment: to a female or male human patient suffering from DI and SU, administer Promensil in the amount of 40-160 mg/day, adjusted according to patient reaction, to alleviate symptoms of incontinence.

### Example 3

Treatment: to a male human patient having benign prostatic hyperplasia affecting continence, administer Promensil in the amount of 40 mg/day.

It will be apparent to those skilled in the art that many obvious modifications and variations may be made to the embodiments described herein without departing from the spirit or the scope of the invention.

### INDUSTRIAL APPLICABILITY

In view of the substantial economic impact of urinary dysfunction, the present invention in its many aspects offers a commercial solution alleviating the problem.

1. A pharmaceutical composition for treatment or preeitlo+f urinary dysfunction in a mammal having lowered purinergic activation, causing partial loss of inhibition of muscaranic receptors, the composition including a pharmaceutically effective amount of one or more substances capable of enabling the bladder of the mammal to mimic condition found in advanced pregnancy.

2. A pharmaceutical composition for treatment or prevention of urinary dysfunction in a mammal having lowered purinergic activation, causing partial loss of inhibition of muscaranic receptors, the composition including a pharmaceutically effective amount of one or more substances adapted to regulate the expression of one or more ATPases that control the supply of ATP to P2X receptors in the bladder of the mammal.

3. The pharmaceutical composition of claim 2, wherein at least one of the ATPases is adapted to control the supply of ATP to the P2X receptors, so as to down-regulate expression of receptor types P2X₁, P2X₂, P2X₃ and P2X₅ in the bladder of the patient.

4. A pharmaceutical composition for treatment or prevent urinary dysfunction in a mammal having lowered purinergic activation, causing partial loss of inhibition of muscaranic receptors, the composition including a pharmaceutically effective amount of one or more substances adapted to down-regulate expression of subtype receptors P2X₁, P2X₂, P2X₃ and P2X₅ in the bladder of the mammal.

5. The pharmaceutical composition of claim 2 or 4, wherein the one or more substances are also adapted to up-regulate expression of subtype receptors P2X₄, and P2X₆.

6. The pharmaceutical composition of claim 2 or 4 wherein the one or more substances is adapted to up-regulate expression of subtype receptors P2X₄, and P2X₆ instead of down-regulating expression of subtype receptors P2X₁, PX2_{2,} PX2₃ and P2X₅.

7. The pharmaceutical composition of any claims 1, 2, 4 and 6, wherein the mammal has lowered purinergic receptors P2X₃ and P2X₅.

8. The pharmaceutical composition of any claims 1, 2, 4 and 6, wherein the mammal has lowered purinergic receptors P2X₁₋₆ and P2X₆.

9. The pharmaceutical composition of any one of claims 1 to 8, wherein the one or more substances are chosen from the group consisting of progestins, estrogens, phytoestrogens and/or isoflavones.

10. The pharmaceutical composition of claim 9, wherein the or at least one of the substances is progesterone.

11. The pharmaceutical composition of claim 9, wherein the or at least one of the substances is a phytoestrogen and/or isoflavone or combination of phytoestrogens.

12. The pharmaceutical composition of claim 9, wherein the or at least one of the substances is a combination of phytoestrogens and/or isoflavones.

13. The pharmaceutical composition of claim 11 or 12, wherein the urinary dysfunction is caused by benign prostate hyperplasia.

14. The pharmaceutical composition of any of one of claims 1 to 13, wherein the urinary dysfunction includes sensory urgency.

15. The pharmaceutical composition of any one of claims 1 to 13, wherein the urinary dysfunction includes detrusor instability.

16. The pharmaceutical composition of any one of claims 1 to 15 wherein the mammal is a human.

17. The pharmaceutical composition of claim 16 when dependant on claim 10, wherein the progesterone is provided in a dose of 50-250 mg/day.

18. The pharmaceutical composition of claim 16 when dependant on claim 11 or 12, wherein the phytoestrogen and/or isoflavone or combination of phytoestrogens and/or isoflavones is provided in a dose of 40-160 mg/day.

19. A method of treating or preventing urinary dysfunction in a mammal, including administrating to the mammal a pharmaceutical composition as defined in any one of claims 1 to 18.

20. Use of a pharmaceutical composition as claimed in anyone of claims 1 to 18, in the treatment or prevention of urinary dysfunction in a mammal having lowered purinergic activation, causing partial loss of inhibition of muscaranic receptors.

21. A pharmaceutical composition substantially as herein described in any of examples 1 to 3 herein.

22. A method of treating or preventing urinary dysfunction in a mammal substantially as herein described in connection with any one of examples 1 to 3 herein.
